(19) United States
(12) Patent Application Publication
(10) Pub. No.: US 2025/0134353 A1
(43) Pub. Date: May 1, 2025

(54) ENDOSCOPE

(71) Applicant: OLYMPUS MEDICAL SYSTEMS CORP., Tokyo (JP)

(72) Inventors: Haruki OKAMOTO, Tokyo (JP); Takanori WATANABE, Tokyo (JP); Yusuke NOMURA, Yokohama-shi (JP)

(73) Assignee: OLYMPUS MEDICAL SYSTEMS CORP., Tokyo (JP)

(21) Appl. No.: 18/918,639

(22) Filed: Oct. 17, 2024

(30) Foreign Application Priority Data
Oct. 25, 2023 (JP) 2023-183067

(51) Int. Cl. A61B 1/00

(52) U.S. Cl. CPC A61B 1/00091; A61B 1/00174

(57) ABSTRACT

An endoscope of an embodiment of the present disclosure includes an observation window and a nozzle at a distal end of an insertion portion. The nozzle configures a flow path having an opening, the opening ejects fluid toward the observation window. The flow path includes a first flow path parallel to a longitudinal direction of the insertion portion and a second flow path communicating with the first flow path and having a curved surface for directing the fluid from the first flow path towards the opening. The curved surface includes a first surface, a second surface and a ridge sandwiched by respective edges of the first surface and the second surface. The respective edges are along a direction of extension of the second flow path.

### CROSS REFERENCE TO RELATED APPLICATION

This application is based upon and claims the benefit of priority from Japanese Patent Application No. 2023-183067 filed in Japan on Oct. 25, 2023, the entire contents of which are incorporated herein by reference.

### BACKGROUND

### 1. Field

The present disclosure relates to an endoscope including a nozzle that ejects fluid on a distal end surface of an insertion portion.

### 2. Description of the Related Art

Endoscopes, which are widely used in the medical field, etc., have an elongated insertion portion to be inserted into a subject. An observation window of an image pickup unit is disposed on a distal end surface of the insertion portion.

Dirt adhering to the observation window obstructs observation. For this reason, a nozzle that ejects fluid is disposed in the vicinity of the observation window on the distal end surface to remove the dirt adhering to the observation window. The nozzle bends by 90 degrees the direction of the flow of the fluid supplied from a flow path in the longitudinal direction of the insertion portion (direction perpendicular to the distal end surface), and ejects the supplied fluid to the observation window.

Japanese Patent Publication No. H11-244221 discloses a nozzle in which the cross-sectional area of a flow path continuously decreases toward an opening and the top surface of the flow path is an inclined circular-arc surface so that the fluid ejected from the nozzle is evenly supplied to the entire observation window.

### SUMMARY

An endoscope of an embodiment of the present disclosure includes an observation window and a nozzle at a distal end of an insertion portion. The nozzle configures a flow path having an opening, the opening ejects fluid toward the observation window. The flow path includes a first flow path parallel to a longitudinal direction of the insertion portion and a second flow path communicating with the first flow path and having a curved surface for directing the fluid from the first flow path towards the opening. The curved surface includes a first surface, a second surface and a ridge sandwiched by respective edges of the first surface and the second surface. The respective edges are along a direction of extension of the second flow path.

### BRIEF DESCRIPTION OF THE DRAWINGS

FIG. 1 is a perspective view of an endoscope system including an endoscope of an embodiment.

FIG. 2 is a front view of a distal end portion of the endoscope of the embodiment.

FIG. 3 is a cross-sectional view of the distal end portion of the endoscope of the embodiment.

FIG. 4 is a cross-sectional view of a nozzle of the endoscope of the embodiment.

FIG. 5A is a cross-sectional view along a line IVA-IVA in FIG. 4.

FIG. 5B is a cross-sectional view along a line IVB-IVB in FIG. 4.

FIG. 5C is a cross-sectional view along a line IVC-IVC in FIG. 4.

FIG. 5D is a cross-sectional view along a line IVD-IVD in FIG. 4.

FIG. 5E is a cross-sectional view along a line IVE-IVE in FIG. 4.

FIG. 5F is a cross-sectional view along a line IVF-IVF in FIG. 4.

FIG. 6 is a perspective view of a flow path of the nozzle of the endoscope of the embodiment.

FIG. 7 is a front view of the flow path of the nozzle of the endoscope of the embodiment.

FIG. 8 is a top view of the flow path of the nozzle of the endoscope of the embodiment.

FIG. 9 is a perspective view of a flow path of a nozzle of an endoscope of a conventional art.

FIG. 10 is a cross-sectional view of a flow path of a nozzle of an endoscope of a modification.

FIG. 11 is a cross-sectional view of a flow path of a nozzle of an endoscope of a modification.

FIG. 12 is a cross-sectional view of a flow path of a nozzle of an endoscope of a modification.

FIG. 13 is a cross-sectional view of a flow path of a nozzle of an endoscope of a modification.

### DETAILED DESCRIPTION

An endoscope 9 of an embodiment is described below with reference to the drawings.

Note that the drawings based on respective embodiments are schematic. The relationship between the thickness and width of each portion, the ratio of the thicknesses, the relative angles, etc., of respective portions differ from those in reality. The drawings also contain parts in which length relationships and ratios are different from each other. Illustrations of some components may be omitted. The distal end side of an insertion portion in the longitudinal direction is referred to as “top”.

### <Endoscope>

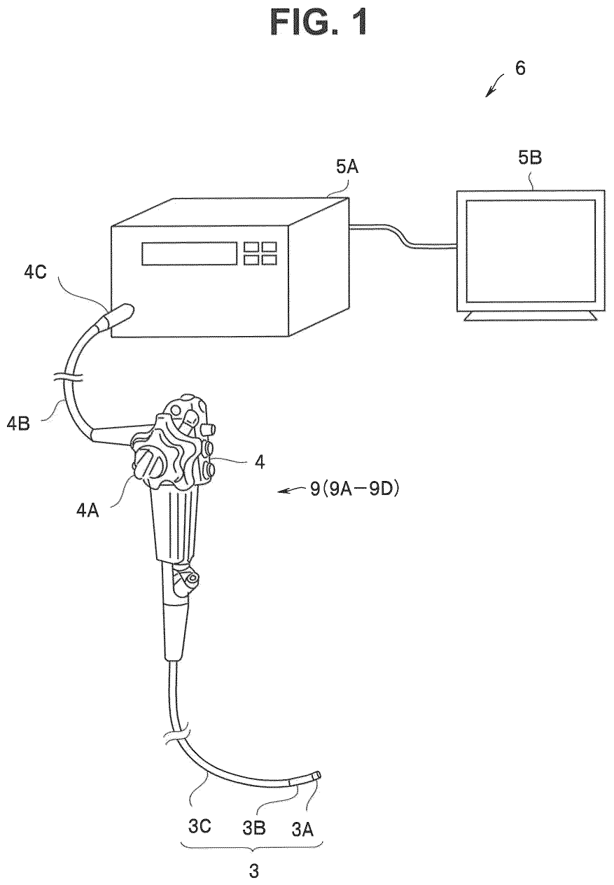

As shown in FIG. 1, the endoscope 9 of the embodiment, together with a processor 5A and a monitor 5B, configures an endoscope system 6.

The endoscope 9 includes an insertion portion 3, a grasping portion 4 disposed at a proximal end portion of the insertion portion 3, a universal cord 4B extending from the grasping portion 4, and a connector 4C disposed at a proximal end of the universal cord 4B. The insertion portion 3 includes a distal end portion 3A, a bending portion 3B extending from the distal end portion 3A, and a flexible portion 3C extending from the bending portion 3B. The bending portion 3B, which is to change the direction of the distal end portion 3A, is bendable. An angle knob 4A, which is for the surgeon to operate the bending portion 3B, is disposed on the grasping portion 4.

The universal cord 4B is connected to the processor 5A by the connector 4C. The processor 5A controls the entirety of the endoscope system 6, performs a signal processing on an image pickup signal from the endoscope, and outputs an image signal. The monitor 5B displays the image signal outputted by the processor 5A as an endoscope image.

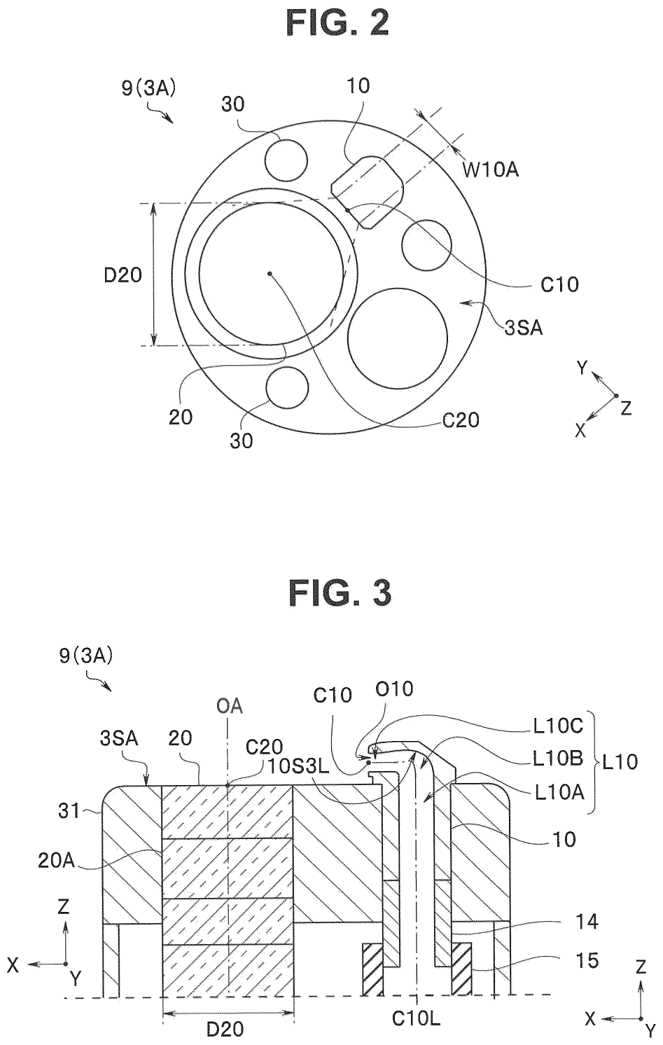

As shown in FIG. 2, a distal end surface 3SA of the distal end portion 3A of the endoscope 9 is provided with a nozzle 10, an observation window 20, and an illumination window 30. As shown in FIG. 3, the observation window 20 is a distal end surface of an optical system 20A that forms a subject image. An optical axis OA of the optical system 20A, which includes a plurality of lenses, is located at a center point C20 of the observation window 20. The subject image formed by the optical system 20A is converted into an image pickup signal by an image pickup device such as a CCD (not shown) and transmitted to the processor 5A. The nozzle 10 configures a flow path L10 having an opening O10 that ejects fluid toward the observation window 20. The nozzle 10 sprays the fluid to clean the observation window 20. The fluid is, e.g., water or air. The illumination window 30 is a distal end surface of the illumination optical system that illuminates a region to be observed.

The observation window 20 is parallel to the distal end surface 3SA and is perpendicular to the optical axis OA. For example, in the XYZ orthogonal coordinate system shown in FIG. 3, etc., a Z axis (top and bottom direction) is the longitudinal direction of the distal end portion 3A of the insertion portion 3. The observation window 20 located in the XY plane is perpendicular to the Z axis. Note that FIG. 3 is a cross-sectional view in the XZ plane, which is a virtual plane, including a center line C10L of the flow path L10, a center point C10 of the opening O10, and the center point C20 of the observation window 20.

As shown in FIG. 3, a distal face of the observation window 20 of the endoscope 9 is located in the same plane as the distal end surface 3SA, but the distal face of the observation window 20 may be surrounded by a ring-shaped slope and protrude from the distal end surface 3SA. A part of the fluid may be ejected from the nozzle 10 toward the slope surrounding the observation window 20.

### <Nozzle>

The nozzle 10 is connected to an air/water feeding tube 15, which passes through the insertion portion 3, by a pipe 14. The nozzle 10 is made of metal or hard resin. The nozzle 10 is produced, e.g., by using a metal cylinder as the base material and forming the flow path L10 by cutting processing. The nozzle 10 may be produced by an injection molding method or produced by using a 3D printer. The nozzle 10 may be configured by combining a plurality of members. A through hole in a distal end rigid member 31 that configures the distal end portion 3A may be used as a part of the flow path L10 of the nozzle 10. A part of the distal end rigid member 31 may be protruded from the distal end surface 3SA to form the nozzle 10.

The flow path L10 includes a first flow path L10A, a second flow path L10B, and a third flow path L10C. The center line C10L of the flow path L10 is a line connecting respective center points G of a plurality of cross sections of the flow path L10.

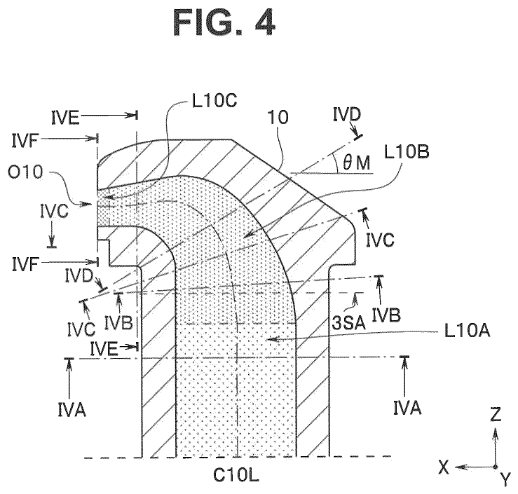

As shown in FIG. 4, the first flow path L10A is parallel to the longitudinal direction (Z axis) of the insertion portion 3, i.e., perpendicular to the distal end surface 3SA. The second flow path L10B is in communication with the first flow path L10A and is curved 90 degrees in the direction of the opening O10. The third flow path L10C is in communication with the second flow path L10B and has the opening O10. The third flow path L10C is perpendicular to the longitudinal direction (Z axis) of the insertion portion 3, i.e., parallel to the distal end surface 3SA. The third flow path L10C may be inclined so that, with increasing distance from a communication portion with the second flow path L10B to the opening O10, an inside inner surface and/or an outside inner surface is closer to the distal end surface 3SA.

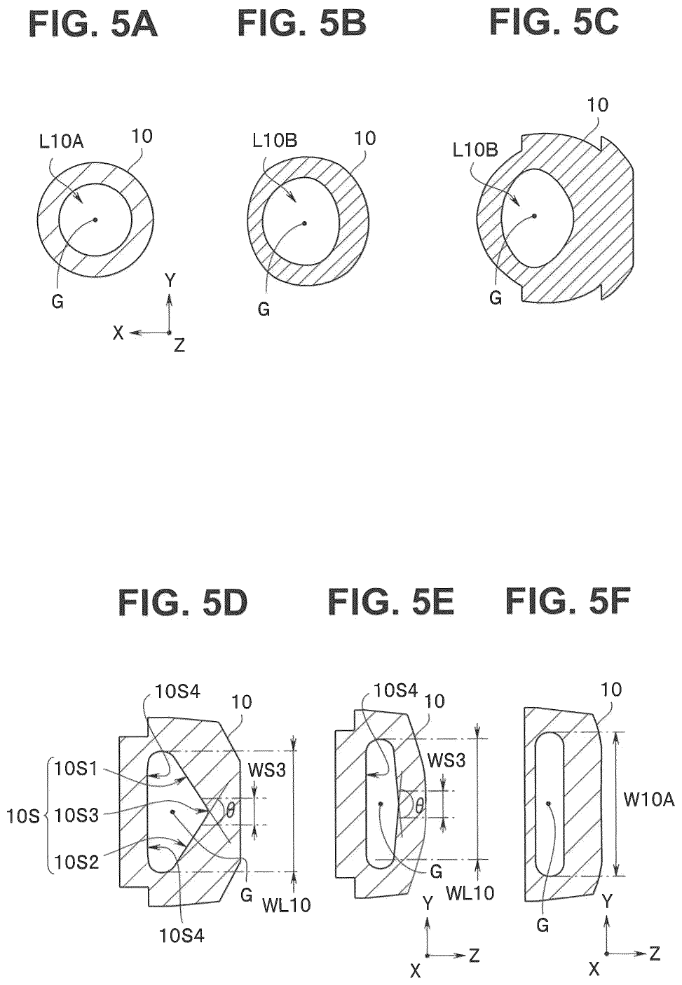

As shown in FIG. 5A, the first flow path L10A has a cross-sectional shape that is a circle. As shown in FIGS. 5B to 5E, the second flow path L10B has a cross-sectional shape that changes continuously. A flow path width WL10 of the flow path L10 has the largest internal dimension in the cross section orthogonal to the center line C10L.

The second flow path L10B includes, e.g., an outside inner surface 10S on the outside in the curvature direction that includes a first surface 10S1, a second surface 10S2, and a third surface 10S3, as shown in FIG. 5D. The first surface 10S1 and the second surface 10S2 are planes.

The third surface 10S3 is sandwiched by the respective edges of the first surface 10S1 and the second surface 10S2, and is an elongated ridge along a direction of extension of the second flow path L10B. A transition between each of the first and second edges and opposing sides of the ridge may be non-continuous.

The third surface 10S3 is a curved surface that is a corner R region. A center line (center line of the ridge) 10S3L of the third surface 10S3 is located on an XZ plane, which is a virtual plane, including the center line C10L of the flow path L10, the center point C10 of the opening O10, and the center point C20 of the observation window 20 (see FIG. 3).

A crossing angle θ between the first surface 10S1 and the second surface 10S2 has a minimum value, e.g., 120 degrees, at the position shown in FIG. 5D. The crossing angle θ increases continuously from the position where the crossing angle θ is minimum (FIG. 5D) toward the opening O10.

The crossing angle θ is 180 degrees at a communication portion with the third flow path L10C. In the communication portion between the second flow path L10B and the third flow path L10C, the first surface 10S1, the second surface 10S2, and the third surface 10S3 are located in the XY plane, and their cross sections configure a single straight line.

As shown in FIG. 5F, the shape of the opening O10 is a track shape, i.e., a substantially rectangle shape having semicircular short sides. In other words, a track shape can also be called an elongated oval shape having a pair of planar opposing sides. The third flow path L10C has a track shape that is the same as the opening O10. The shape of the opening O10 may be a substantially rectangle shape having the four corners of the rectangle shape with a corner R.

The flow path L10 may not include the third flow path L10C. That is, the second flow path L10B may have the opening O10.

The position where the crossing angle θ is minimum (FIG. 5D) is a curvature middle position where the flow path is curved significantly, i.e., a position where a line IVD-IVD intersects the distal end surface 3SA at an angle θM (see FIG. 4) of greater than 30 degrees and less than 60 degrees, as shown in FIG. 4.

The crossing angle θ has a maximum value (180 degrees) at a position proximate to the opening O10, a minimum value at the significantly curved position shown in FIG. 5D, and increases with decreasing distance to the opening O10, as shown in FIG. 5E.

The minimum value of the crossing angle θ can be greater than 60 degrees and less than 150 degrees. If the minimum value of the crossing angle θ is within this range, the nozzle 10 is able to efficiently eject fluid in the width direction of the opening O10 without increasing the outer dimensions of the nozzle 10.

Note that the second flow path L10B cannot be said to have the three distinct surfaces (the first surface 10S1, the second surface 10S2, and the third surface 10S3) in a region proximate to the first flow path L10A. In other words, it is difficult to distinguish the three surfaces (the first surface 10S1, the second surface 10S2, and the third surface 10S3). However, the second flow path L10B has the three surfaces on the outside inner surface 10S that is on the outside in the curvature direction, at least from the position where the crossing angle θ is the minimum toward the opening O10.

Note that from at least the position in the second flow path L10B where the crossing angle θ is minimum to the third flow path L10C, a fourth surface 10S4, which is the inside inner surface that is on the inside in the curvature direction, is a plane. The first surface 10S1 and the second surface 10S2 are connected to the fourth surface 10S4 via curved surfaces.

At least in the vicinity of the third surface 10S3, the first surface 10S1 and the second surface 10S2, if planes, may include a curved surface.

The cross-sectional shape of the third flow path L10C orthogonal to a flow path center line has a track shape that is the same as the opening O10. In other words, the fourth surface 10S4 is a plane.

To place the nozzle on the distal end surface 3SA that is narrow, an opening width W10A of the opening O10 of the nozzle 10 is small, e.g., 50% of an outer diameter D20 of the observation window 20. The opening width W10A is the maximum dimension in a Y-axis direction in a direction orthogonal to the center line C10L, i.e., a direction orthogonal to the XZ plane, which is a virtual plane.

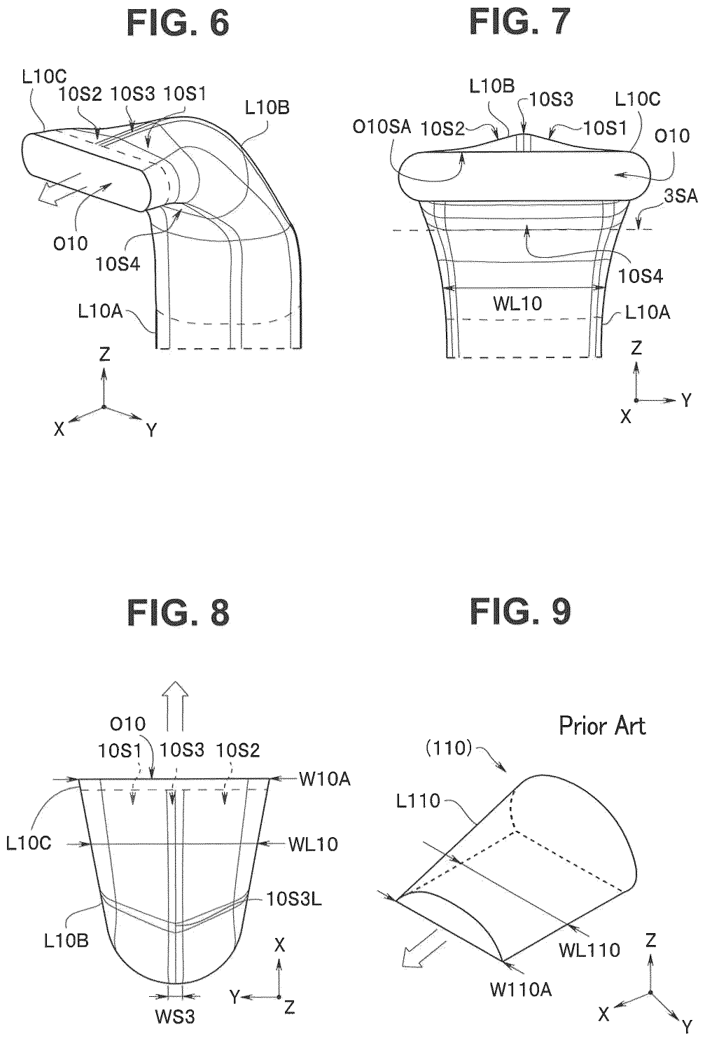

FIGS. 6 to 8 show a shape of an internal space of the nozzle 10, i.e., a shape of the flow path L10. As shown in FIGS. 6 and 7, the fluid that passes through the first flow path L10A is curved by 90 degrees in the direction of flow while ascending along the second flow path L10B, and via the third flow path L10C, is ejected out in a direction substantially parallel to the distal end surface 3SA.

In the endoscope 9, in which the area of the distal end surface 3SA is small, the opening width W10A of the opening O10 of the nozzle 10 is, e.g., 50% of the outer diameter D20 of the observation window 20. In order to eject fluid over the entire surface of the observation window 20, the second flow path L10B of the nozzle 10 has the flow path width WL10 that increases continuously toward the opening O10.

In other words, as shown in FIG. 8, the flow path width WL10 of the second flow path L10B increases continuously from at least the position where the crossing angle θ is minimum to the communication portion with the third flow path L10C. The flow path width WL10 of the third flow path L10C also increases continuously from the communication portion with the second flow path L10B to the opening O10. In this case, the third flow path L10C is considered a part of the second flow path L10B. In other words, the flow path L10 may not include the third flow path L10C.

For this reason, the fluid ejected from the opening O10 is ejected over a wide range. In order to eject the fluid over a wide range, the opening width W10A can be greater than 10% and less than 70% of the outer diameter D20 of the observation window 20.

Note that the flow path width WL10 of the third flow path L10C may be constant. In this case, the fluid is ejected to a narrower area than in a case where the flow path width WL10 increases.

Note that, although the cross-sectional shapes of the first flow path L10A, the second flow path L10B, and the third flow path L10C differ significantly, the cross-sectional area of flow path L10 does not increase or decrease significantly. The largest cross-sectional area of the flow path L10 can be less than 150% and, alternatively, can be less than 125%, of the minimum cross-sectional area of the flow path L10. If the cross-sectional area is less than these values, the flow path L10 has a small pressure drop, allowing the nozzle 10 to eject the fluid efficiently.

As shown in FIG. 7, the flow path width WL10 increases continuously in a region of the first flow path L10A proximate to the second flow path L10B. The flow path width WL10 of the nozzle 10 increases continuously toward the opening O10 and is at the maximum at the opening O10. The flow path width WL10 of the flow path L10 changes smoothly in a curved shape in all the regions. In endoscopes of conventional arts, the flow path width widens only in the flow path near an opening. In contrast, the endoscope of this embodiment has a gradual increase in the flow path width over a longer distance, making it difficult for turbulent flow to occur.

In the absence of the third surface 10S3 that is a ridge, when fluid is ejected from the nozzle 10 of the opening O10 over a wider range than the opening width W10A, the velocity of the ejected flow is higher at an end portion and lower at a center portion.

However, in the second flow path L10B of the nozzle 10, the outside inner surface 10S on the outside of the curvature direction has the third surface 10S3, which is a ridge and elongated. The outside inner surface 10S of the second flow path L10B protrudes to the top side with respect to the top end surface O10SA of the opening O10. The fluid ascending in the flow path 10L generates a fast flow along the third surface 10S3 that is narrow and located at the top center of the flow path L10. This fast flow reaches the center point C20 of the observation window 20, efficiently removing dirt adhering near the center point C20.

Note that a ridge width WS3, which is the width of the elongated third surface (ridge) 10S3 in the direction orthogonal to the XZ plane, which is a virtual plane, is smaller than the outer diameter D20 of the observation window 20, and therefore the flow tends to concentrate on the third surface 10S3. Furthermore, if the ridge width WS3 is less than 30% of the flow path width WL10 of the second flow path L10B, the flow is even more likely to concentrate on the third surface 10S3. The third surface 10S3 may be a plane if the ridge width WS3 is less than 30% of the flow path width WL10 of the second flow path L10B.

In the nozzle 10, the ridge width WS3 is substantially the same over the entire range of the second flow path L10B, but may change. For example, the ridge width WS3 may continuously decrease toward the opening O10 if the ridge width WS3 is less than 30% of the flow path width WL10 of the second flow path L10B at least at the position where the crossing angle θ is minimum.

FIG. 9 shows a shape of a flow path L110 of a nozzle 110 disclosed in Japanese Patent Application No. H11-244221. The top surface of the cross section of the flow path L110 is a circular arc, and a flow path width WL110 is unchanged. The flow path width WL110 is the same as the opening width W110A of the nozzle 110. In the flow path L110, the opening width W110A of the nozzle 110 is large and has the same outer diameter as the observation window, which enables the nozzle 110 to eject fluid over the entire surface of the observation window. However, the nozzle 110 does not have an elongated third surface, causing the fluid to flow in a less concentrated manner. Reducing the opening width makes the fluid to flow in a more concentrated manner, but is not able to efficiently eject the fluid over a wide range. In addition, because the cross-sectional area of the flow path L110 is significantly reduced, the nozzle 110 has a large pressure drop.

In contrast, the nozzle 10 of this embodiment has a small pressure drop and able to efficiently eject fluid over a wider range than the opening width W10A.

### MODIFICATIONS OF EMBODIMENT

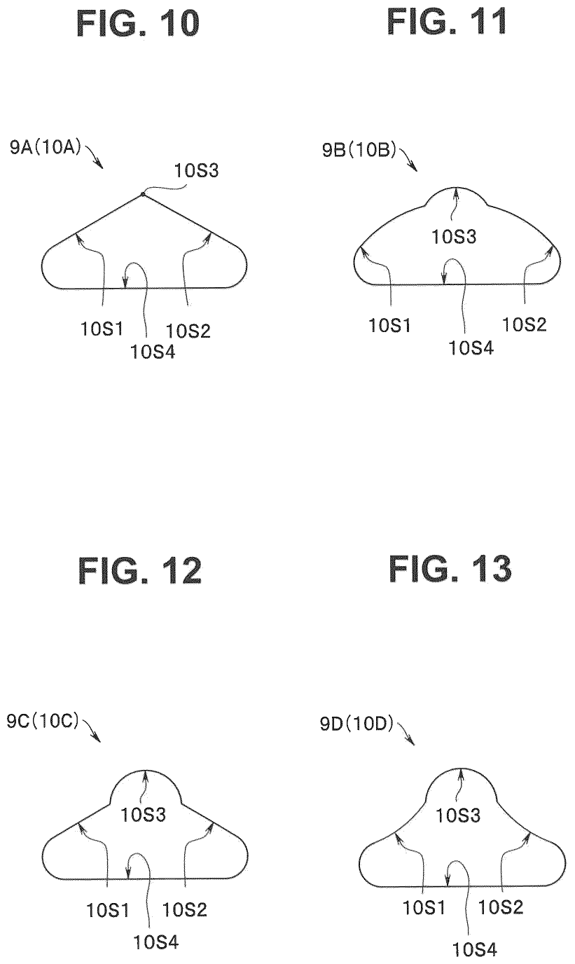

Next, endoscopes 9A-9D of modifications 1-3 of the embodiment will be described. Since the endoscopes 9A-9D are similar to the endoscope 9 and have the same effects as the endoscope 9, components with the same functions as those of the endoscope 9 are marked with the same numerals and the descriptions are omitted. For example, FIGS. 10 to 13 are each a cross-sectional view of the flow path 10L at a position where the crossing angle θ is minimum.

### Modification 1 of Embodiment

As shown in FIG. 10, in the endoscope 9A of this modification, the first surface 10S1 and the second surface 10S2 are planes, and a crossing line (i.e., an apex) between the first surface 10S1 and the second surface 10S2 is considered a ridge for the purposes of this disclosure. The crossing line (ridge) is one as seen from an industrial point of view, which if enlarged, is a corner having a radius R with a width in the Y direction, even if such width may be considered minimal. For example, a corner R having the ridge width WS3 that is less than 0.1 mm may be a line but is nonetheless considered herein as a ridge.

### Modification 2 of Embodiment

As shown in FIG. 11, in an endoscope 9B of this modification, the first surface 10S1 and the second surface 10S2 are curved surfaces that are each convex relative to a center line of the flow path. The curvature (reciprocal of the radius of curvature) of the curved surfaces of the first surface 10S1 and the second surface 10S2 decreases continuously from the position where the crossing angle θ is minimum toward the opening O10. The curvature of the third surface 10S3, which is a curved surface sandwiched between the first surface 10S1 and the second surface 10S2, is greater than the curvature of the first surface 10S1 (second surface 10S2).

### Modification 3 of Embodiment

As shown in FIG. 12, in an endoscope 9C of this modification, the first surface 10S1 and the second surface 10S2 are planes, and the third surface 10S3, which is sandwiched between the first surface 10S1 and the second surface 10S2, is a curved surface.

### Modification 4 of Embodiment

As shown in FIG. 13, in an endoscope 9D of this modification, the first surface 10S1 and the second surface 10S2 are curved surfaces that are each concave relative to a center line of the flow path, and the third surface 10S3, which is sandwiched between the first surface 10S1 and the second surface 10S2, is a curved surface that is convex at the top.

The endoscope 9 may be a rigid endoscope in which the insertion portion 3a is rigid. The endoscope 9 may be used for medical or industrial purposes. The present disclosure is not limited to the above-described embodiments, etc., and various changes, modifications, etc. can be made within the scope that does not change the gist of the disclosure.

What is claimed is:

1. An endoscope including:
an observation window; and
a nozzle at a distal end of an insertion portion, wherein
the nozzle configures a flow path having an opening, the opening ejects fluid toward the observation window,
the flow path includes:
a first flow path parallel to a longitudinal direction of the insertion portion; and
a second flow path communicating with the first flow path and having a curved surface for directing the fluid from the first flow path towards the opening, and
the curved surface including:
a first surface;
a second surface; and
a ridge sandwiched by respective edges of the first surface and the second surface, and the respective edges being along a direction of extension of the second flow path.

2. The endoscope according to claim 1, wherein a center line of the ridge is located on a virtual plane including a center line of the flow path, a center point of the opening, and a center point of the observation window.

3. The endoscope according to claim 2, wherein an opening width of the opening is greater than 10% and less than 70% of an outer diameter of the observation window.

4. The endoscope according to claim 2, wherein a minimum value of a crossing angle between the first surface and the second surface is greater than 60 degrees and less than 150 degrees.

5. The endoscope according to claim 4, wherein the crossing angle increases in the second flow path between the first flow path and the opening.

6. The endoscope according to claim 5, wherein a flow path width of the second flow path increases between the first flow path and the opening.

7. The endoscope according to claim 1, wherein
the ridge having a curved surface, and
a ridge width of the ridge is smaller than an outer diameter of the observation window.

8. The endoscope according to claim 7, wherein the ridge width is less than 30% of a flow path width of the second flow path.

9. The endoscope according to claim 1, wherein the ridge is an apex of the first surface and the second surface.

10. The endoscope according to claim 6, wherein the first surface and the second surface are each planar.

11. The endoscope according to claim 6, wherein
the first surface and the second surface are curved surfaces having a same shape, and
a curvature of the curved surfaces of the first surface and the second surface decreases continuously between the first flow path and the opening.

12. The endoscope according to claim 6, wherein the flow path width of the flow path increases continuously in the second flow path.

13. The endoscope according to claim 6, wherein the opening has an elongated oval shape having a pair of planar opposing sides.

14. The endoscope according to claim 6, wherein an inside inner surface of the flow path in a curvature direction is a plane from the position where the crossing angle is minimum toward the opening.

15. The endoscope according to claim 6, further comprising:
a third flow path that is in communication with the second flow path, wherein
the third flow path includes the opening,
a cross section of the third flow path orthogonal to a center line has an elongated oval shape having a pair of planar opposing sides, and
the flow path width of the third flow path is constant.

16. The endoscope according to claim 1, wherein the first surface and the second surface are each concave relative to a center line of the flow path.

17. The endoscope according to claim 1, wherein the first surface and the second surface are each convex relative to a center line of the flow path.

18. The endoscope according to claim 1, wherein the first surface having a first edge, the second surface having a second edge, a transition between each of the first and second edges and opposing sides of the ridge being non-continuous.

19. The endoscope according to claim 1, wherein the ridge is convex relative to a center line of the flow path.

20. The endoscope according to claim 1, wherein
the first surface and the second surface are each convex relative to a center line of the flow pat;
the ridge is convex relative to a center line of the flow path; and
a radius of curvature of the ridge is greater than a radius of curvature of each of the first and second surfaces.
